(19) United States
(12) Patent Application Publication
(10) Pub. No.: US 2011/0071410 A1
(43) Pub. Date: Mar. 24, 2011

(54) CUFF FOR HEMADYNAMOMETER

(75) Inventor: Cha Ki Chul, Seoul (KR)

(73) Assignee: BIOSPACE CO., LTD., Seoul (KR)

(21) Appl. No.: 12/781,758

(22) Filed: May 17, 2010

(30) Foreign Application Priority Data
Sep. 21, 2009 (KR) 10-2009-0089105

(51) Int. Cl. A61B 5/022

(52) U.S. Cl. 600/499

(57) ABSTRACT

Disclosed is a cuff for a hemadynamometer, which is used for measuring blood pressure of a testee. The cuff includes a fluid bag for applying pressure to a body of the testee, at least one flexible member disposed at an outer portion of the fluid bag and a cover member accommodating the fluid bag and the flexible member therein. The flexible member is maintained in a stretched state and is deformed into an annular state when a force is applied to the flexible member in a winding direction of the cuff such that the cuff is wound around the testee. The cuff can be wound around the testee, so that blood pressure is measured. A testee is able to wind the cuff around his or her own arm without the need for assistance.

### CROSS REFERENCE TO RELATED APPLICATION(S)

This application claims the benefit under 35 U.S.C. § 119(a) of Korean Patent Application No. 10-2009-0089105, filed on Sep. 21, 2009, the entire disclosure of which is incorporated herein by reference for all purposes.

### BACKGROUND

1. Field

The following description relates to a cuff for a hemadynamometer, which is wound around a testee to measure blood pressure.

2. Description of the Related Art

In general, in order to measure blood pressure, a cuff for a hemadynamometer including a fluid bag is wound around an arm of a testee and the fluid bag is repeatedly inflated and deflated to detect pulse waves in an arterial tube. The cuff includes a cover member serving as a fastening strip. The cuff can be fastened to the arm of the testee by means of the cover member. Thus, the cover member must be reliably wound around the arm of the testee such that the fluid bag can be fixed to the arm of the testee when the cuff is used.

However, in the case of a conventional cuff for the hemadynamometer, the testee winds the cuff around his or her own arm, so the cuff may not be reliably wound around the arm. If the cuff is not reliably wound, inaccuracies may occur in the measurement value of the hemadynamometer, so the blood pressure may not be precisely and stably measured.

In order to reliably wind the cuff around the arm of the testee, as shown in FIG. 1, a flexible member called a “curler” may be accommodated in the cover member of the cuff for the hemadynamometer (see, Japanese unexamined patent publication No. 2003-210423).

Referring to FIG. 1, a curler 11 made of flexible material is provided along an outer surface of a fluid bag. Due to the curler 11, a cuff 10 can be maintained in an annular configuration and can be elastically deformed in a radial direction. In the case of the cuff 10 having the curler 11, the fluid bag can be fixedly pressed against the arm due to the curler 11, so the cuff 10 can be reliably wound around the arm of the testee.

However, in the case of the cuff 10 for the hemadynamometer including the curler 11, the cuff 10 having the annular configuration must be widened in order to wind the cuff 10 around the arm of the testee, so that attaching of the cuff 10 is complicated, and the testee alone may not easily wind the cuff 10 around his or her own arm.

In order to solve the above problem, as shown in FIGS. 2A to 2C, Japanese unexamined patent publication No. 2002-209858 discloses a cuff for a hemadynamometer, which can be easily used. Referring to FIGS. 2A to 2C, the cuff includes a curler 21 which is wound in an annular configuration and is stretchable outward. Thus, when the testee uses the cuff, the testee winds the curler 21 around his or her arm 31 while unwinding the curler 21 from the annular configuration by stretching the curler 21 into a state in which one end of the curler 21 is placed on the arm 31, thereby facilitating the winding of the cuff around the arm of the testee.

However, this case also requires force to unwind and stretch the curler 21. In particular, in the case of old, infirm, and weak persons, a person with limited mobility or reaching distance, and some women, it may be difficult to widen the curler 21. In addition, in the case of a user who has a big upper arm, a high force is required to widen the curler 21, so difficulty of attaching the cuff is increased and the cuff may not be precisely wind around the arm of the testee.

According to the related art, as shown in FIGS. 1 and 2, the position of the cuff may be changed when the cuff is wound around the upper arm by widening the curler 11 or 21, so that the position of the sensor may deviate from the measurement position. For this reason, the user must check the position of the sensor and adjust the position of the cuff to correct the position of the sensor, causing inconvenience to the user. According to another related art, the sensor is additionally inserted between the upper arm and the cuff after the cuff has been wound around the upper arm. However, in this case, the sensor inserting step is additionally required, causing inconvenience to the user, and further opportunity for an inaccurate attachment and setup of the device to occur.

In addition, in order to improve measurement precision, a sensor installed in the cuff must be precisely located in the measurement position when the cuff is wound around the arm. However, when the cuff is wound around the arm 31 while widening the curler, the sensor may not be located in the precise position.

Further, the cuff may move along the arm when the cuff is wound around the arm. In this case, the user must check the sensor position and move the cuff to locate the sensor in the measurement position, thereby causing inconvenience to the user. Although the sensor may be inserted between the arm and the cuff after the cuff has been wound around the arm, this requires an additional and sometimes difficult step for inserting the sensor.

### SUMMARY

Accordingly, in one aspect, there is provided a cuff for a hemadynamometer, in which the cuff can be precisely wound around an arm of a testee so that blood pressure can be precisely and stably measured and in which an old, infirm, or weak person, a person with limited mobility or reaching distance, or a testee who has a big upper arm can reliably wind the cuff around his or her own arm without the need for assistance.

In order to accomplish the above object, according to an embodiment, there is provided a cuff for a hemadynamometer, which is wound around an arm of a testee. The cuff includes a fluid bag for applying pressure to the arm, at least one flexible member disposed at an outer portion of the fluid bag, and a cover member accommodating the fluid bag and the flexible member therein, wherein the flexible member is maintained in a stretched state and is deformed into an annular state when a force is applied to the flexible member in a winding direction of the cuff such that the cuff is wound around the arm of the testee.

As described above, according to an embodiment, an old, weak, or infirm person, a person with limited mobility or reaching distance, a woman, or a testee who has a big upper arm can reliably wind the cuff around the arm without great effort. In addition, the testee can easily, reliably and precisely wind the cuff around his or her own arm without the need for assistance.

According to an embodiment, since the cuff can be maintained in a stretched state when the cuff is not used, the cuff may occupy a smaller space when the cuff is not used, so the cuff can be easily stored.

According to an embodiment, adjusting the position of the cuff to adjust the sensor position and inserting an additional sensor after the cuff is wound around the arm of the testee are not necessary, and the sensor can be precisely positioned in the measurement position when the cuff is wound around the arm. Thus, the measurement precision can be improved and the blood pressure can be precisely and stably measured.

According to an embodiment, a plurality of flexible members are provided while being spaced apart from each other in the width direction of the cuff, so that fastening force of the cuff with respect to the upper arm can be adjusted.

According to an embodiment, flexible members having relatively strong elastic force are disposed at both sides of the cuff and flexible members having relatively weak elastic force are disposed at the center of the cuff, so edge portions of the cuff can be securely fastened to the upper arm regardless of the curvature of the upper arm.

According to an embodiment, the flexible members apply relatively strong elastic force to the front and rear ends of the cuff while applying relatively weak elastic force to the center of the cuff, so that the cuff can be securely wound around the upper arm of the testee.

In one general aspect, there is provided a cuff for a hemadynamometer, which is wound around a body of a testee, the cuff including: a fluid bag configured to applying pressure to the body, at least one flexible member disposed at an outer portion of the fluid bag, and a cover member accommodating the fluid bag and the flexible member therein, wherein the flexible member: is maintained in a stretched state, and is deformed into an annular state when a force is applied to the flexible member in a winding direction of the cuff such that the cuff is wound around the body of the testee.

The cuff may further include: a plurality of flexible members, wherein the plurality of flexible members are spaced apart from each other in a width direction of the cuff.

The cuff may include that the plurality of flexible members includes at least three flexible members, in which flexible members positioned at each edge of the cuff have a greater elastic recovery force as compared with a flexible member positioned at a center of the cuff in the annular state.

The cuff may include that each flexible member includes a greater elastic recovery force at end portions thereof as compared with a center thereof in the annular state.

The cuff may include that: the flexible member includes a property which allows the stretched state and the annular state to be maintained, and the states of the flexible member are interchanged when a force having a value exceeding a critical value is applied to the flexible member.

The cuff may further include: a plurality of flexible members, wherein the plurality of flexible members are spaced apart from each other in a width direction of the cuff.

The cuff may include that the plurality of flexible members includes at least three flexible members, in which flexible members positioned at each edge of the cuff have a greater elastic recovery force as compared with a flexible member positioned at a center of the cuff in the annular state.

The cuff may include that each flexible member includes a greater elastic recovery force at end portions thereof as compared with a center thereof in the annular state.

The cuff may include that, when a force having a value less than a critical value is applied to the flexible member arranged in the annular state, the flexible member maintains the annular state due to an elastic recovery force.

The cuff may further include: a plurality of flexible members, wherein the plurality of flexible members are spaced apart from each other in a width direction of the cuff.

The cuff may include that the plurality of flexible members includes at least three flexible members, in which flexible members positioned at each edge of the cuff have a greater elastic recovery force as compared with a flexible member positioned at a center of the cuff in the annular state.

The cuff may include that each flexible member includes a greater elastic recovery force at end portions thereof as compared with a center thereof in the annular state.

Other features and aspects will be apparent from the following detailed description, the drawings, and the claims.

### BRIEF DESCRIPTION OF THE DRAWINGS

FIG. 1 is a perspective view showing a conventional cuff for a hemadynamometer;

FIGS. 2A to 2C are a perspective view showing the procedure for winding a conventional cuff for a hemadynamometer around an arm;

FIG. 3 is a perspective view showing a cuff for a hemadynamometer according to an embodiment;

FIG. 4 is a perspective view showing a contact surface of a cuff for a hemadynamometer shown in FIG. 3;

FIG. 5 is a partially-cut perspective view of a cuff for a hemadynamometer shown in FIG. 4;

FIG. 6 is a sectional view taken along line A-A of FIG. 5;

FIGS. 7 to 9 are views showing the procedure for winding a cuff for a hemadynamometer shown in FIG. 3 around an arm;

FIG. 10 is a perspective view showing a stretched state and an annular state of a flexible is member shown in FIG. 3;

FIG. 11 is a plan view of flexible members shown in FIG. 3 according to the first embodiment; and

FIG. 12 is a plan view of flexible members shown in FIG. 3 according to the second embodiment.

Elements, features, and structures are denoted by the same reference numerals throughout the drawings and the detailed description, and the size and proportions of some elements may be exaggerated in the drawings for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. The progression of processing steps and/or operations described is an example; however, the sequence of steps and/or operations is not limited to that set forth herein and may be changed as is known in the art, with the exception of steps and/or operations necessarily occurring in a certain order. Also, descriptions of well-known functions and constructions may be omitted for increased clarity and conciseness.

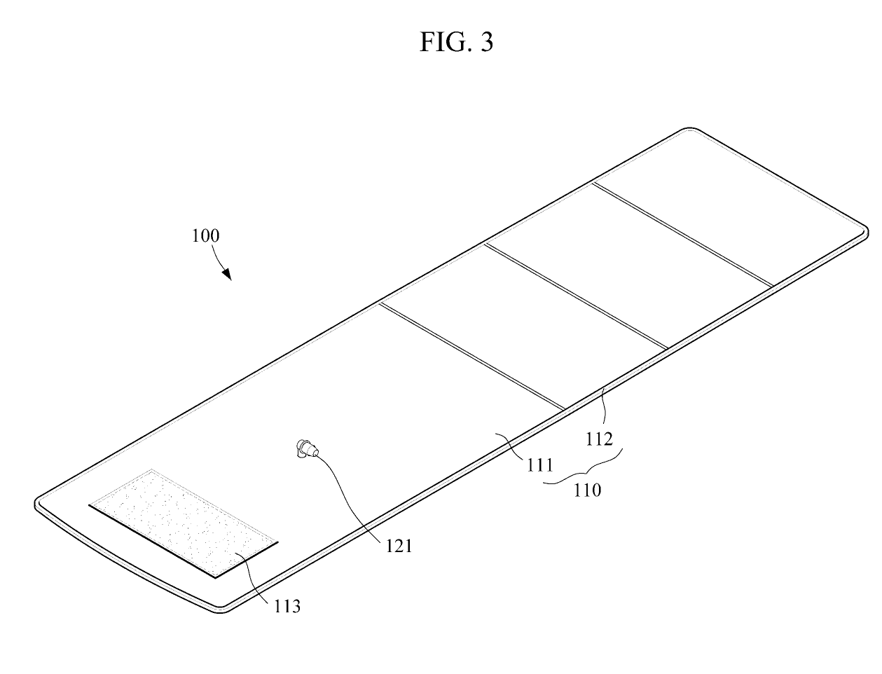

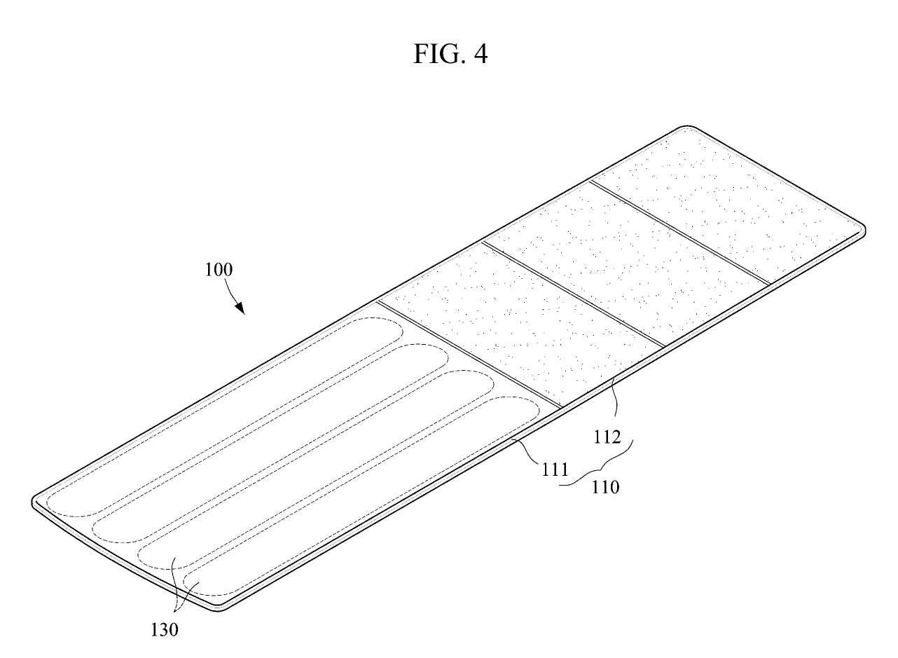

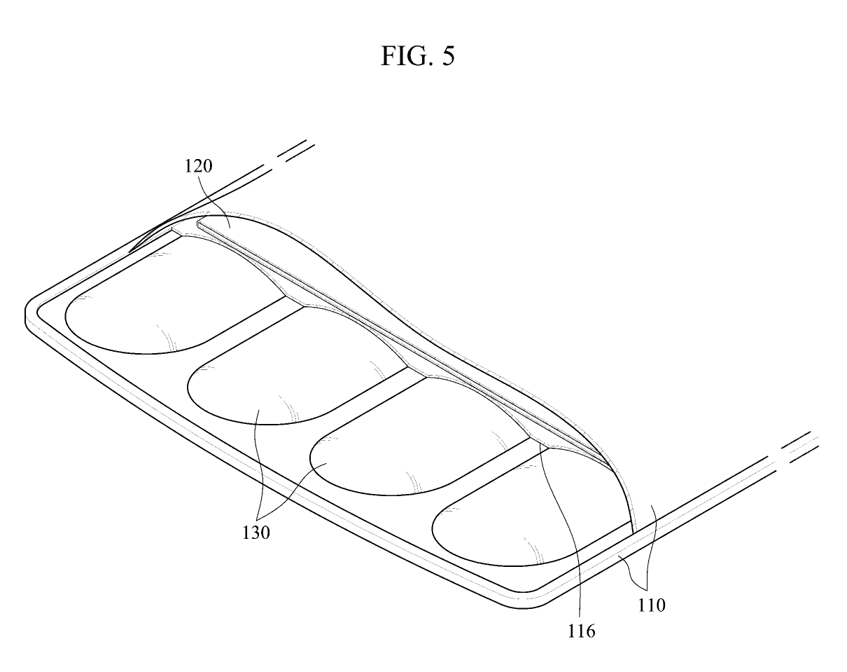

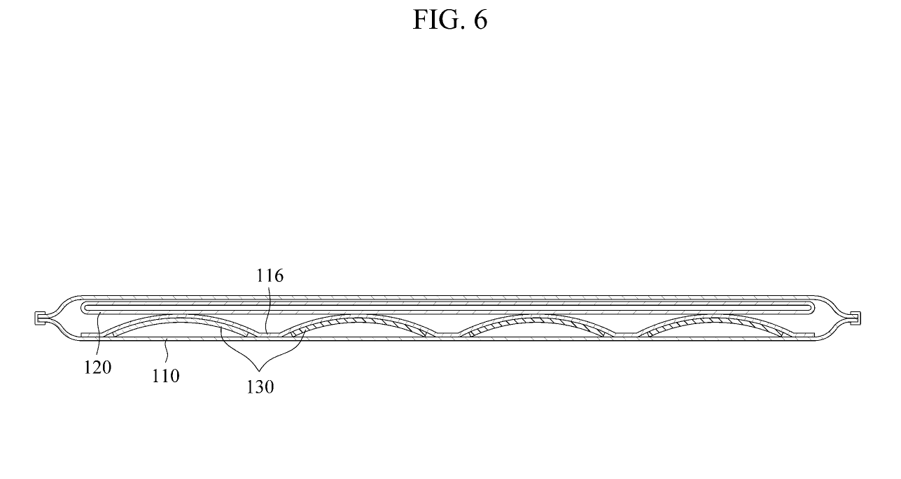

FIG. 3 is a perspective view showing a cuff for a hemadynamometer according to an embodiment, FIG. 4 is a perspective view showing a contact surface of the cuff for the hemadynamometer shown in FIG. 3, FIG. 5 is a partial cut-out perspective view of the cuff for the hemadynamometer shown in FIG. 4, and FIG. 6 is a sectional view taken along line A-A of FIG. 5. For the purpose of convenience in explanation, a surface of the cuff making contact with the arm of the testee will be referred to as an inner surface, and the opposite surface not making contact with the arm will be referred to as an outer surface.

Referring to FIGS. 3 to 6, the cuff 100 for the hemadynamometer is wound around a body of a testee, for example, an upper arm of the testee. The cuff 100 includes a cover member 110, a fluid bag 120, and at least one flexible member 130.

The cover member 110 forms an outer skin of the cuff 100 and may be made of a cloth, or the like. The cover member 110 may have a pocket shape in which the fluid bag 120 and the flexible member 130 may be accommodated. The cover member 110 may include a pressing part 111, in which the fluid bag 120 and the flexible member 130 may be accommodated, and a fixing part 112 extending from the pressing part 111. A fastener 113 may be attached to an outer surface of the fixing part 112. The fixing part 112 may surround the upper arm in a state in which the pressing part 111 is wound around the upper arm. At this time, the pressing part 111 may be fastened by the fastener 113, so that the cuff 100 can be stably wound around the upper arm.

The fluid bag 120 may be accommodated in the cover member 110 to press the upper arm of the testee. The fluid bag 120 may have an internal space for receiving fluid, such as air. The internal space of the fluid bag 120 may be communicated with a rubber tube (not shown) through a nipple 121. The nipple 121 may protrude outward from an outer surface of the cover member 110. In addition, although not shown in the drawings, the rubber tube may be connected to a boosting pump and a negative pressure pump accommodated in a body of the hemadynamometer.

When the blood pressure is measured, air may be introduced into or withdrawn from the internal space of the fluid bag 120 by the boosting pump and the negative pressure pump, so that the fluid bag 120 may be inflated or deflated. When the fluid bag 120 is inflated, the upper arm of the testee may be pressed. A sensor 101 may be installed at an inner surface of the fluid bag 120. The sensor 101 may detect systolic pressure and diastolic pressure when the fluid bag 120 is inflated and deflated during the measurement of the blood pressure. The sensor 101 may be fixed to the cover member 110 in the position corresponding to the center of the fluid bag 120.

The flexible member 130 may be provided at the side of the outer surface of the fluid bag 120. The flexible member 130 may be arranged in a stretched state. In this state, when external force is applied to the flexible member 130 in the winding direction of the cuff 100, the flexible member 130 may be deformed in an annular shape, so that the cuff 100 can be wound around the upper arm of the testee. In addition, if an external force is applied to the flexible member 130 in the direction opposite to the winding direction of the cuff 100, the flexible member 130 can return to the stretched state. When the flexible member 130 is arranged in the annular state, the fluid bag 120 can be fastened to the upper arm. In addition, when flexible member 130 is arranged in the stretched state, the fluid bag 120 can be released from the upper arm.

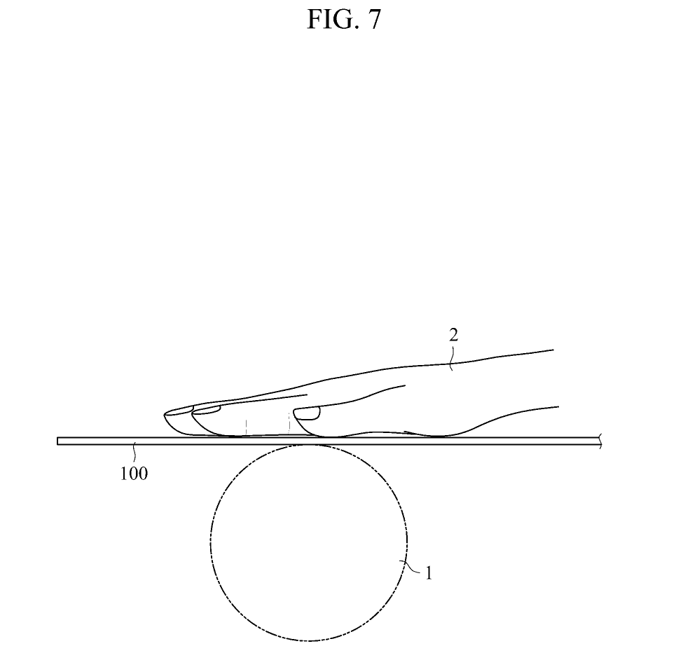

Hereinafter, the operation of the cuff 100 for the hemadynamometer having the above structure will be described with reference to FIGS. 7 to 9. In the following description, the cuff 100 may be wound around a left upper arm 1, but the cuff 100 can also be wound around a right upper arm.

First, as shown in FIG. 7, the user may arrange the cuff 100 in the stretched state by flattening the flexible member 130. In this state, the user may place the pressing part 111 of the cover member 110 on the left upper arm 1 by using his/her right hand 2. At this time, the pressing part 111 may be located such that the sensor 101 can be positioned corresponding to the artery of the left upper arm 1. Then, after touching the outer surface of the pressing part 111 using the palm of his/her right hand 2, the user may grip the pressing part 111 while applying force to the pressing part 111 by using the fingers of his/her right hand 2.

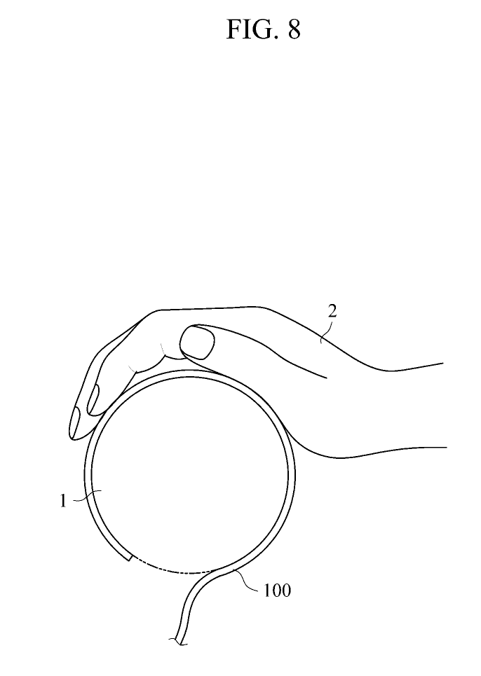

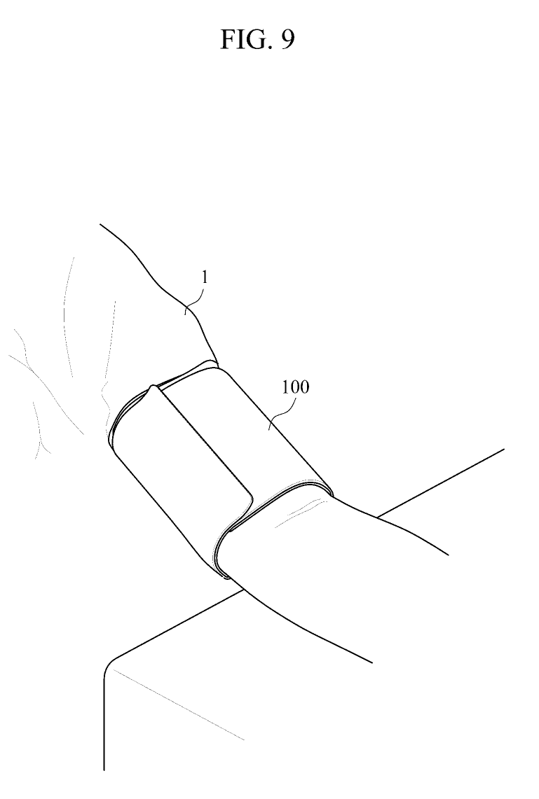

As a result, the flexible member 130 may be deformed into the annular shape so that the pressing part 111 may be wound around the left upper arm 1 as shown in FIG. 8. At this time, since the flexible member 130 is arranged in the annular state, the pressing part 111 can be maintained in the winding state with respect to the left upper arm 1. Subsequently, the user may hold the fixing part 112 of the cover member 110 using the right hand 2 and may overlap the fixing part 112 over the pressing part 111. Then, the user may fix the fixing part 112 to the pressing part 111 by using the fastener 113. As a result, as shown in FIG. 9, the cuff 100 can be wound around the left upper arm 1. The cuff 100 can be released from the left upper arm 1 by performing the above procedure in reverse.

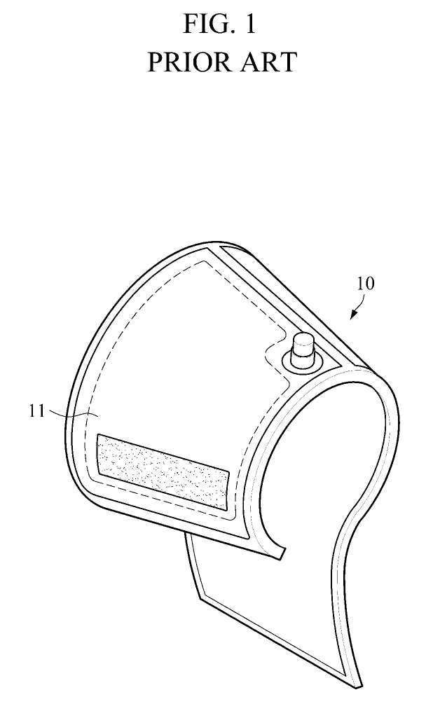

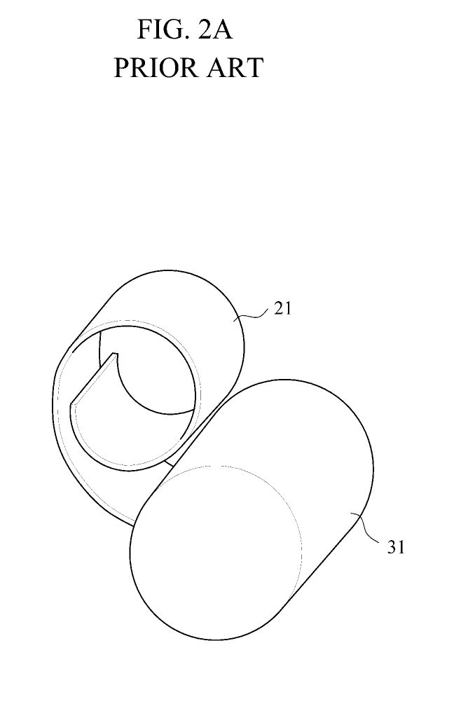

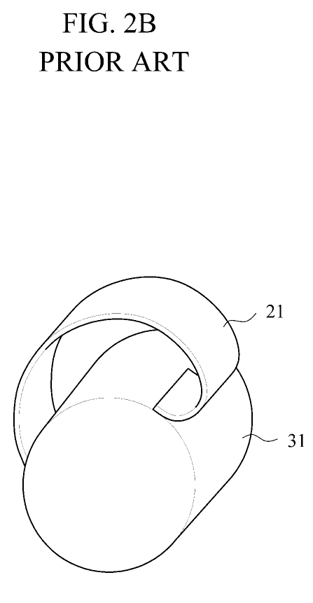

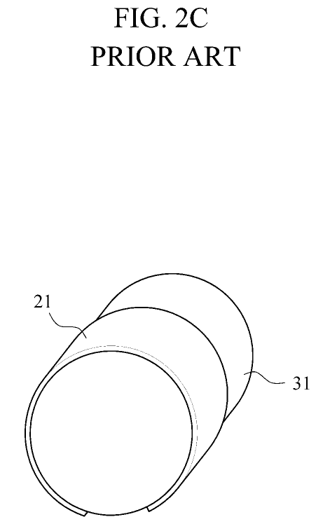

As mentioned above, in a state in which the cuff 100 is arranged in the stretched state, the cuff 100 may be placed in the precise position and the user may wind the cuff 100 around the upper arm by applying force to the cuff 100. Therefore, different from the related art shown in FIGS. 1 and 2, in which the user winds the cuff around the upper arm while widening the curler 11 or 21 using one hand, old, infirm, and weak people, a person with limited mobility or reaching distance, some women, or testees who have big upper arms can reliably wind the cuff 100 around the upper arm without applying great force. In addition, the user can easily and precisely wind the cuff 100 around the upper arm by oneself.

According to an embodiment, since the cuff 100 is arranged in the stretched state, the user can place the cuff 100 in the precise position on the upper arm after adjusting the position of the cuff 100 such that the sensor 101 can be positioned corresponding to the artery of the upper arm. Therefore, it may not be necessary to correct the position of the cuff 100 after the cuff 100 has been wound around the upper arm and the step of inserting an additional sensor is not required. In addition, the sensor 101 can be precisely located in the measurement position when the cuff 100 is wound around the upper arm. Thus, the blood pressure can be reliably and stably measured with high precision.

Furthermore, according to an embodiment, since the cuff 100 can be arranged in the stretched state when the cuff 100 is not used, the cuff 100 may occupy a smaller space when the cuff 100 is not used, so the cuff 100 can be easily stored.

Meanwhile, the flexible member 130 can be stably maintained in either one of the stretched state and the annular state. These states of the flexible member 130 can be interchanged if a force having a value above a critical value is applied to the flexible member 130. In addition, if a force having a value less than the critical value is applied to the flexible member 130 arranged in the annular state, the annular state of the flexible member 130 is not changed due to the elastic recovery properties of the flexible member 130.

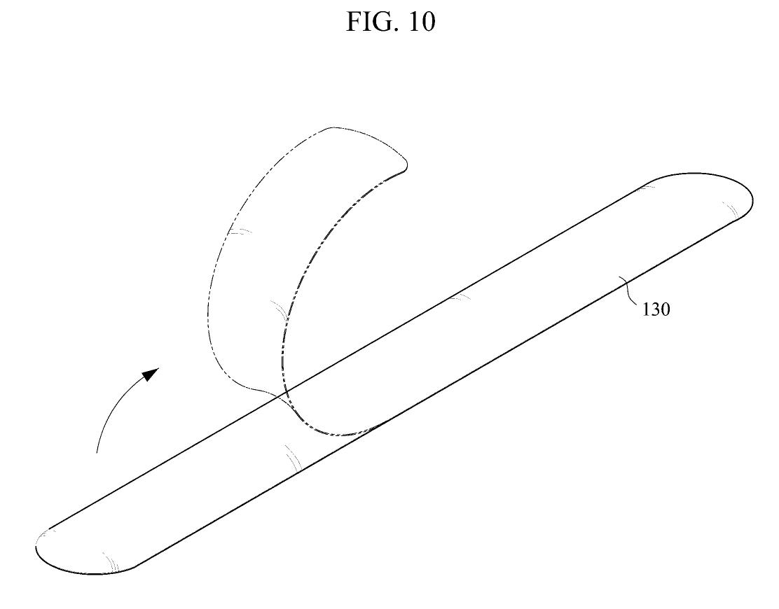

For instance, referring to FIG. 10 as well as FIGS. 6 and 7, when the flexible member 130 is in the stretched state, the flexible member 130 may extend lengthwise along the cuff 100 in a plate shape and while being curved having a circular arc cross sectional shape which is convex toward the inner surface of the cuff 100.

Since the flexible member 130 may be rolled in the width direction such that the flexible member 130 has the sectional shape of the circular arc, the flexible member 130 can be maintained in the stretched state. When the flexible member 130 is arranged in the stretched state, if a force having a value less than the critical value is applied to the flexible member 130 arranged in the stretched state, the stretched state of the flexible member 130 may not be changed due to the elastic recovery properties of the flexible member 130. The force having critical value may correspond to the force that deforms the sectional shape of the circular arc.

If a force capable of deforming the sectional shape of the circular arc is applied to the flexible member 130, the flexible member 130 arranged in the strectched state may be urged to be changed into the annular state, so that the flexible member 130 may be arranged in the annular state. In this state, if a force that is insufficient to recover the sectional shape of the circular arc of the flexible member 130 is applied to the flexible member 130, the flexible member 130 can maintain the annular state due to the elastic recovery properties of the flexible member 130.

Based on the above principle, the flexible member 130 may be arranged in the stretched state and then may be changed into the annular state when the force is applied to the cuff 100 in the winding direction so that the cuff 100 can be wound around the upper arm. Thus, the cuff 100 can be wound around the upper arm even if a relatively weak force is applied to the flexible member 130, so that the user can easily wind the cuff 100 around the upper arm.

Meanwhile, a plurality of flexible members 130 can be provided while being spaced apart from each other in the width direction of the cuff 100. Thus, when the cuff 100 has been wound around the upper arm of the testee, a pressing force for the upper arm may be generated only in the positions where the flexible members 130 are located and the pressing force for the upper arm is not generated in the gap between the flexible members 130. Therefore, the pressing force of the cuff 100 with respect to the upper arm can be adjusted by changing the gap between the flexible members 130.

The flexible members 130 may be fixedly interposed between the cover member 110 and a position fixing cloth 116. The position fixing cloth 116 can be seamed to the cover member 110 such that the flexible members 130 can be fixedly arranged while being spaced apart from each other. Although the flexible members 130 are physically separated from each other in the drawings, the flexible members 130 can be integrally formed with each other.

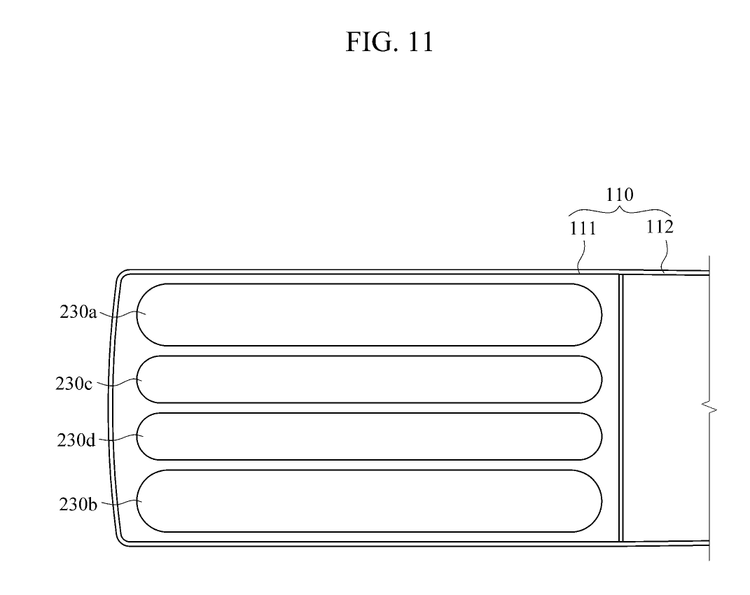

As shown in FIG. 11, four flexible members 230a to 230d can be provided. In this case, the flexible members 230a and 230b positioned at both sides of the cover member 110 may have greater elastic recovery force as compared with the flexible members 230c and 230d positioned at the center of the cover member 110. To this end, the flexible members 230a and 230b positioned at both sides of the cover member 110 may have larger widths than that of the flexible members 230c and 230d positioned at the center of the cover member 110 such that the flexible members 230a and 230b may have a greater elastic recovery force in the annular state. The four flexible members 230a to 230d may be designed to have the same critical value such that the four flexible members 230a to 230d can be simultaneously deformed into the stretched state or the annular state.

When the four flexible members 230a to 230d are provided, the flexible members 230a and 230b having relatively greater elastic recovery force may be positioned at both sides of the cuff 100 and the flexible members 230c and 230d having relatively weaker elastic recovery force may be positioned at the center of the cuff 100. Thus, edge portions of the cuff 100 can be securely fastened to the upper arm of the user even if the user has the great curvature in the upper arm due to having a large bicep. Although an example embodiment may include four flexible members 230a to 230d, embodiments are not limited thereto. For example, three or five flexible members can be provided within the scope of embodiments.

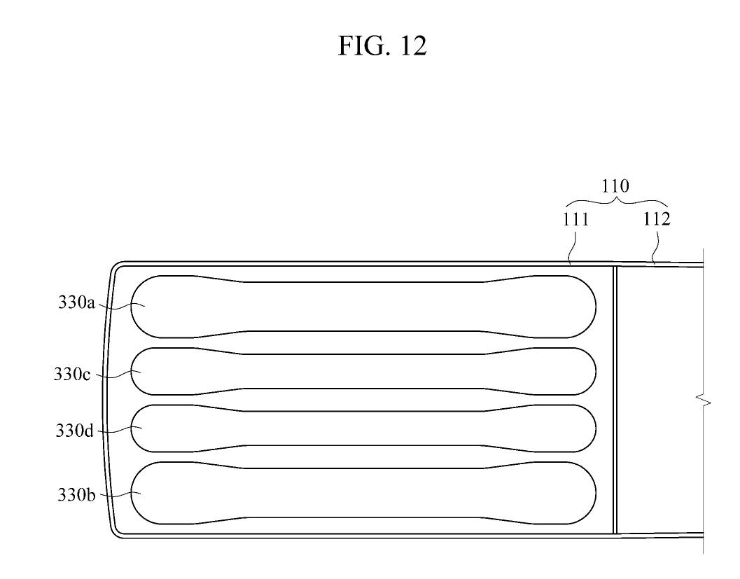

As shown in FIG. 12, flexible members 330a to 330d can be provided such that greater elastic recovery force can be generated at both end portions of the flexible members 330a to 330d as compared with the center portions of the flexible members 330a to 330d. To this end, the flexible members 330a to 330d may have larger widths at both end portions thereof as compared with the center portions thereof such that the side portions of the flexible members 330a to 330d can have greater elastic recovery force in the annular state.

In this case, the flexible members 330a to 330d have relatively strong elastic recovery force at the front and rear ends of the pressing part 111 as compared with the center of the pressing part 111. Thus, when the cuff 100 is wound around the upper arm, the front and rear ends of the pressing part 111 may be more fastened, so that the cuff 100 can be securely wound around the upper arm. Meanwhile, the flexible members may have the same size.

A number of example embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A cuff for a hemadynamometer, which is wound around a body of a testee, the cuff comprising:
a fluid bag configured to applying pressure to the body;
at least one flexible member disposed at an outer portion of the fluid bag; and
a cover member accommodating the fluid bag and the flexible member therein,
wherein the flexible member:
is maintained in a stretched state; and
is deformed into an annular state when a force is applied to the flexible member in a winding direction of the cuff such that the cuff is wound around the body of the testee.

2. The cuff of claim 1, further comprising:
a plurality of flexible members,
wherein the plurality of flexible members are spaced apart from each other in a width direction of the cuff.

3. The cuff of claim 2, wherein the plurality of flexible members comprises at least three flexible members, in which flexible members positioned at each edge of the cuff have a greater elastic recovery force as compared with a flexible member positioned at a center of the cuff in the annular state.

4. The cuff of claim 2, wherein each flexible member comprises a greater elastic recovery force at end portions thereof as compared with a center thereof in the annular state.

5. The cuff of claim 1, wherein:
the flexible member comprises a property which allows the stretched state and the annular state to be maintained; and
the states of the flexible member are interchanged when a force having a value exceeding a critical value is applied to the flexible member.

6. The cuff of claim 5, further comprising:
a plurality of flexible members,
wherein the plurality of flexible members are spaced apart from each other in a width direction of the cuff.

7. The cuff of claim 6, wherein the plurality of flexible members comprises at least three flexible members, in which flexible members positioned at each edge of the cuff have a greater elastic recovery force as compared with a flexible member positioned at a center of the cuff in the annular state.

8. The cuff of claim 6, wherein each flexible member comprises a greater elastic recovery force at end portions thereof as compared with a center thereof in the annular state.

9. The cuff of claim 1, wherein, when a force having a value less than a critical value is applied to the flexible member arranged in the annular state, the flexible member maintains the annular state due to an elastic recovery force.

10. The cuff of claim 9, further comprising:
a plurality of flexible members,
wherein the plurality of flexible members are spaced apart from each other in a width direction of the cuff.

11. The cuff of claim 10, wherein the plurality of flexible members comprises at least three flexible members, in which flexible members positioned at each edge of the cuff have a greater elastic recovery force as compared with a flexible member positioned at a center of the cuff in the annular state.

12. The cuff of claim 10, wherein each flexible member comprises a greater elastic recovery force at end portions thereof as compared with a center thereof in the annular state.
